(19) United States
(12) Patent Application Publication
(10) Pub. No.: US 2011/0060165 A1
(43) Pub. Date: Mar. 10, 2011

(54) METAL AMINOTROPONIMINATES, BIS-OXAZOLINATES AND GUANIDINATES

(75) Inventors: Thomas M. Cameron, Newtown, CT (US); Chongying Xu, New Milford, CT (US); Tianniu Chen, Rocky Hill, CT (US); Matthias Stender, New Milford, CT (US)

(73) Assignee: ADVANCED TECHNOLOGY MATERIALS, INC., Danbury, CT (US)

(21) Appl. No.: 12/517,901

(22) PCT Filed: Dec. 29, 2006
(86) PCT No.: PCT/US06/62713

§ 371 Date: Jun. 26, 2009

Related U.S. Application Data
(60) Provisional application No. 60/868,564, filed on Dec. 5, 2006.

(51) Int. Cl. C07C 249/00; C09D 7/12; C23C 16/00

(52) U.S. Cl. 564/248; 106/1.25; 427/255.394

(57) ABSTRACT

Metal aminotroponiminates, metal bis-oxazolinates and metal guanidinates are described, as well as ligand precursors of such compounds, and mixed ligand barium and strontium complexes suitable for chemical vapor deposition, atomic layer deposition, and rapid vapor deposition processes. Such metal compounds are useful in the formation of thin metal films on substrates, e.g., in chemical vapor deposition, atomic layer deposition or rapid vapor deposition processes. The substrates formed have thin film monolayers of the metals provided by the precursors.

### CROSS-REFERENCE TO RELATED APPLICATION

The benefit of priority of U.S. Provisional Patent Application 60/868,564 filed Dec. 5, 2006 is hereby claimed.

### FIELD OF THE INVENTION

The invention relates generally to metal source precursors and their synthesis. In one aspect, the invention relates to strontium, barium and other metal aminotroponiminates, strontium, barium and other bis-oxazolinates, strontium and barium guanidinates, as well as metal guanidinates including metals other than strontium and barium, and methods of making and using these compositions. The invention in another aspect relates to ligand precursors of the inventive metal source precursors. The invention also relates to mixed ligand copper complexes suitable for chemical vapor deposition, atomic layer deposition and rapid vapor deposition applications. In a still further aspect, the invention relates to methods of depositing metal layers on a substrate utilizing the precursors of the invention and substrates generated thereby.

### BACKGROUND OF THE INVENTION

Chemical vapor deposition (CVD) is a chemical process that involves a series of chemical reactions to produce a thin layer of solid material on a substrate surface. The process is widely used to fabricate microelectronic devices and products.

In a typical CVD process, a substrate is exposed to one or more precursors. The precursors react with the substrate surface to produce a deposit of solid material on such surface. CVD is well-suited to provide uniform coverage of the deposited material on the substrate.

Atomic layer deposition (ALD) is a modified CVD process involving a sequential step technique that results in a coating of multiple layers on the substrate. Typically ALD is carried out utilizing two complementary precursors that are alternately introduced to the reaction chamber. The first precursor is delivered in excess into the deposition chamber. The precursor will react with the substrate to form a monolayer of reacted precursor on the surface. The deposition chamber is purged or evacuated with a carrier gas to remove unreacted precursor followed by the delivery of a reactant (a second precursor) to the deposition chamber for reaction with the monolayer of reacted precursor, to form the desired material. This cycle is repeated until an appropriate thickness of material is achieved. Advantageously, ALD provides uniform step coverage and a high level of control over film thicknesses.

In an illustrative ALD process, sequential precursor pulses are used to form a film, layer by layer. A first precursor may be introduced to form a gas monolayer on a substrate, followed by introduction of a second precursor to react with the gas monolayer to form a first solid monolayer of the film. Each cycle including first and second precursor pulses therefore forms one solid monolayer. The process then is repeated to form successive layers until a film of desired thickness is obtained.

An additional deposition process is rapid vapor deposition (RVD). In RVD, similar to ALD, the substrate is sequentially exposed to precursors in gaseous form. In RVD the process is repeated until a substrate coated with multiple layers reaches a desired thickness. The resulting coated substrate is of high conformality. RVD differs from ALD in that the layers in RVD can be deposited more quickly.

Liquid precursors and/or solid precursors dissolved in suitable solvents enable the direct injection and/or liquid delivery of precursors into a CVD, ALD or RVD vaporizer unit. The accurate and precise delivery rate can be obtained through volumetric metering to achieve reproducibility during CVD, ALD or RVD metallization of a VLSI device. Solid precursor delivery via specially-designed devices, such as ATMI's ProE Vap® precursor storage and dispensing package or liquid precursor delivery via specially-designed devices, such as ATMI's NOWTrak® precursor storage and dispensing package (both from ATMI, Inc., Danbury, Conn., USA) enables highly efficient transport of solid precursors to a CVD, ALD or RVD reactor.

Historically, deposition of strontium or barium materials using ALD techniques has been performed utilizing precursor complexes that have a high (>300° C.) transport temperature and provide non-conformal substrate surface coverage. Thus it is desirable to develop new precursors for delivery of barium or strontium with transport temperatures specific to the ALD and/or RVD processes and that promote conformal film production. Efficient and economic methods of making and using such precursors would also be desirable.

Recently, guanidinate anions have received attention for use as ligands in coordination and organometallic compounds, specifically because of the ease of substitution at the carbon and nitrogen atoms and the consequent versatility and flexibility that is provided. Use of such ligands has been limited to lithium salts of the general formula R₁N═C(NR₂R₃)NR₄Li. Complexes including guanidinate ligands are formed by reaction of the corresponding carboiimide (R₁N═C═NR₄) and appropriate LiNR₂R₃ reagent. Development of alternative guanidinate compounds would enable the synthesis of a larger range of guanidinate-containing metal source precursors and would therefore be desirable. Methods of making and using such precursors in a cost-effective and efficient manner would also be desirable.

### SUMMARY OF THE INVENTION

The present invention relates to metal source precursors for use in CVD, ALD and RVD processes and methods of making the same, as well as to a method of depositing a metal layer on a substrate using such precursors and to substrate structures, e.g., microelectronic device structures, having such layers deposited thereon. The invention also relates to ligand precursors useful in making metal source precursors for CVD, ALD and RVD processes.

In one aspect, the invention relates to a ligand precursor selected from the group consisting of compounds of formulas:

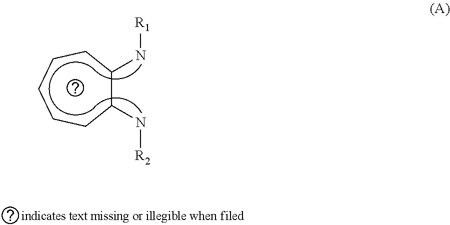

where R₁ and R₂ may be the same as or different from one another and each is independently selected from the group consisting of: H, C₁-C₅ alkyls, C₆-C₁₀ aryls and C₃-C₆ cycloalkyls;

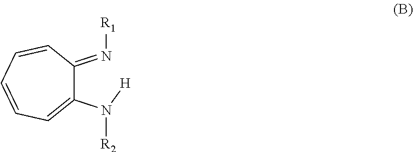

where R₁ and R₂ may be the same as or different from one another and each is independently selected from the group consisting of: H, C₁-C₅ alkyls, C₆-C₁₀ aryls and C₃-C₆ cycloalkyls;

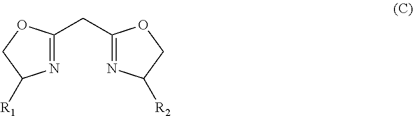

where R₁ and R₂ may be the same as or different from one another and each is independently selected from the group consisting of: H, C₁-C₅ alkyls, C₆-C₁₀ aryls and C₃-C₆ cycloalkyls;

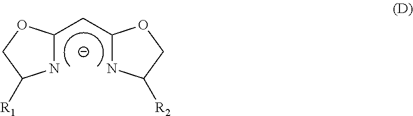

where R₁ and R₂ may be the same as or different from one another and each is independently selected from the group consisting of: H, C₁-C₅ alkyls, C₆-C₁₀ aryls and C₃-C₆ cycloalkyls;

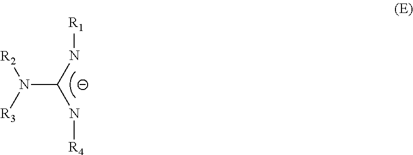

where R₁, R₂, R₃ and R₄ may be the same as or different from one another and each is independently selected from the group consisting of: H, C₁-C₅ alkyls, C₆-C₁₀ aryls and C₃-C₆ cycloalkyls;

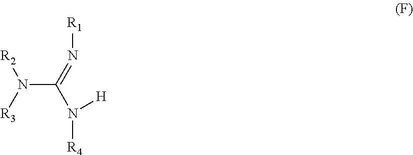

where R₁, R₂, R₃ and R₄ may be the same as or different from one another and each is independently selected from the group consisting of: H, C₁-C₅ alkyls, C₆-C₁₀ aryls and C₃-C₆ cycloalkyls; and

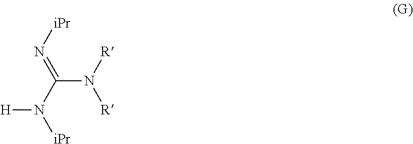

wherein R′ may be the same or different from one another and may be methyl or iPr.

In another aspect, the invention relates to a metal source precursor selected from the group consisting of compounds of the formulas:

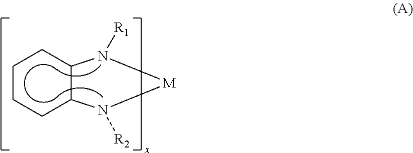

where R₁ and R₂ may be the same as or different from one another and each is independently selected from the group consisting of: H, C₁-C₅ alkyls, C₆-C₁₀ aryls and C₃-C₆ cycloalkyls; M is selected from the group consisting of Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te; and x is 1 to 8, dependent on the oxidation state of M. In one aspect of this embodiment, the metal deposited is selected from the group consisting of Ba and Sr;

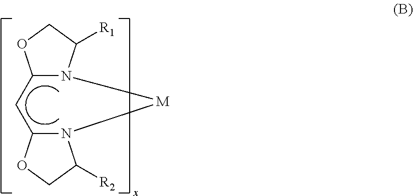

where each of the R₁ and R₂ substituents may be the same as or different from one another and each is independently selected from the group consisting of: H, C₁-C₅ alkyls, C₆-C₁₀ aryls and C₃-C₆ cycloalkyls; M is selected from the group consisting of Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te; and x and x is 1 to 8, dependent on the oxidation state of M. In one aspect of this embodiment, the metal deposited is selected from the group consisting of Ba and Sr;

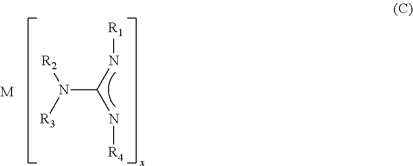

where R₁, R₂, R₃ and R₄ may be the same as or different from one another and each is independently selected from the group consisting of: H, C₁-C₅ alkyls, C₆-C₁₀ aryls and C₃-C₆ cycloalkyls; M is selected from the group consisting of Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te; and x is 1 to 8, dependent on the oxidation state of M. In one aspect of this embodiment, the metal deposited is selected from the group consisting of Ba and Sr;

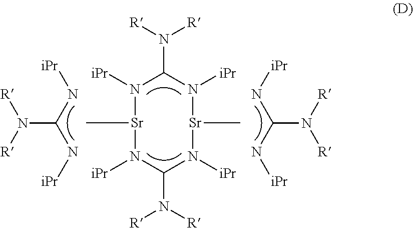

wherein R′ may be the same or different from one another and may be methyl or iPr;

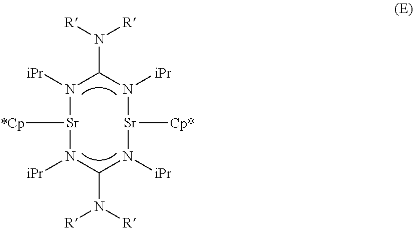

wherein R′ may be the same or different from one another and may be methyl or iPr and wherein Cp* is pentamethylcyclopentadienyl;

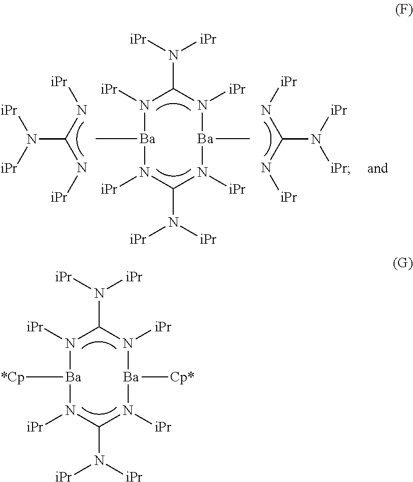

wherein Cp* is pentamethylcyclopentadienyl.

In additional aspects, the invention relates to various methods of making the above metal source precursors.

In still another aspect, the invention relates to a method of depositing a metal layer on a substrate comprising deposit of the metal on the substrate surface by ALD or RVD, wherein at least one precursor is selected from the metal source precursors of the invention. In one aspect the metal deposited is selected from the group comprising Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te. In another embodiment the one or more layers comprise strontium and/or barium. In one embodiment of this method, the ALD is performed at a temperature of less than or equal to 300 degrees Celsius. In another aspect of the embodiment, delivery of the at least one precursor is by solution delivery. In still another aspect of the embodiment, delivery of the at least one precursor is by dispensing from a ProE Yap® precursor storage and dispensing package. In still another aspect, delivery of the at least one precursor involves dispensing from a NOWTrak® precursor storage and dispensing package.

In still another embodiment, the invention relates to a substrate coated with one or more film monolayers of one or more metals. The substrate of the invention is coated by chemical vapor deposition, atomic layer deposition and/or rapid vapor deposition and the deposition method utilizes one or more metal source precursors of the invention. In one embodiment of the invention the one or more layers comprise Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and/or Te. In another embodiment the one or more layers comprise strontium and/or barium.

In yet another aspect of the invention, the invention relates to a ligand precursor selected from the group consisting of compounds of the formulas:

R₁N═C(NR₂R₃)NR₄M (A)

where R₁, R₂, R₃ and R₄ may be the same as or different from one another and each is independently selected from the group consisting of: H, C₁-C₅ alkyls, C₆-C₁₀ aryls and C₃-C₆ cycloalkyls; and M is selected from the group consisting of Na and K; and

R₁N═C(NR₂R₃)NR₄H (B)

where R₁, R₂, R₃ and R₄ may be the same as or different from one another and each is independently selected from the group consisting of: H, C₁-C₅ alkyls, C₆-C₁₀ aryls and C₃-C₆ cycloalkyls.

A further aspect of the invention relates to a metal source precursor of the formula M[R₁N═C(NR₂R₃)NR₄]ₓ, wherein R₁, R₂, R₃ and R₄ may be the same as or different from one another and each is independently selected from the group consisting of: H, C₁-C₅ alkyls, C₆-C₁₀ aryls and C₃-C₆ cycloalkyls; M is selected from the group consisting of Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te; and x is the oxidation state of M.

Yet another aspect of the invention relates to a method of depositing a metal layer on a substrate comprising deposit of the metal on the substrate surface by atomic layer deposition or rapid layer deposition, wherein at least one precursor is a metal source precursor of the formula M[R₁N═C(NR₂R₃)NR₄]ₓ, wherein R₁, R₂, R₃ and R₄ may be the same as or different from one another and each is independently selected from the group consisting of: H, C₁-C₅ alkyls, C₆-C₁₀ aryls and C₃-C₆ cycloalkyls; M is selected from the group consisting of Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te; and x is the oxidation state of M. In one aspect of this embodiment, the metal deposited is selected from the group consisting of Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te. In a further embodiment of this aspect of the invention, the ALD is performed at a temperature of less than or equal to 300 degrees Celsius. In another embodiment of this aspect of the invention, delivery of the at least one precursor is by solution delivery. In still another embodiment of this aspect of the invention, delivery of the at least one precursor is by dispensing from a ProE Vap® precursor storage and dispensing package. In still another aspect, delivery of the at least one precursor involves dispensing from a NOWTrak® precursor storage and dispensing package.

In still another embodiment, the invention relates to a substrate coated with one or more film monolayers of one or more metals. The substrate of the invention is coated by chemical vapor deposition, atomic layer deposition and/or rapid vapor deposition and the deposition method utilizes one or more metal source precursors of the invention. In one embodiment of the invention, the deposition method comprises use of at least one precursor selected from metal source precursors of the formula M[R₁N═C(NR₂R₃)NR₄]ₓ, wherein R₁, R₂, R₃ and R₄ may be the same as or different from one another and each is independently selected from the group consisting of: H, C₁-C₅ alkyls, C₆-C₁₀ aryls and C₃-C₆ cycloalkyls; M is selected from the group consisting of Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te; and x is the oxidation state of M.

The invention in another aspect relates to a precursor storage and delivery apparatus comprising a vessel containing a metal source precursor of the invention.

A further aspect of the invention relates to a vapor of a metal source precursor of the invention.

A still further aspect of the invention relates to a method of making a microelectronic device product, comprising contacting a microelectronic device substrate with a metal source precursor of the invention, to deposit said metal on the substrate.

Yet another aspect relates to a mixed ligand barium and strontium complexes suitable for use in CVD, ALD and RVD applications. Such mixed ligand copper complexes have the general formula:

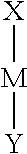

wherein M is barium or strontium, X and Y are each monoanionic and selected from the parent ligands (A)-(H) below, with the proviso that X and Y are different from one another:
(A) Triazacyclononane-amide (tacn) Ligands of the Formula

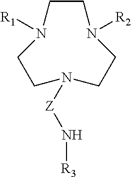

wherein: Z is (CH₂)₂ or SiMe₂; and R₁, R₂ and R₃ are the same as or different from one another, and each is independently selected from among C₁-C₅ alkyl, C₆-C₁₀ aryl, and C₃-C₆ cycloalkyl;

### (B) Aminotroponimine Ligands of the Formula

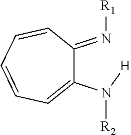

wherein R₁, R₂ are the same as or different from one another and each is independently selected from among H, C₁-C₅ alkyl, C₆-C₁₀ aryl, and C₃-C₆ cycloalkyl;

### (C) Bis(oxazole) Ligands of the Formula

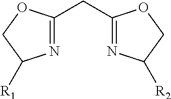

wherein R₁, R₂ are the same as or different from one another and each is independently selected from among H, C₁-C₅ alkyl, C₆-C₁₀ aryl, and C₃-C₆ cycloalkyl;

### (D) Guanidine Ligands of the Formula

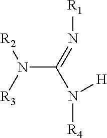

wherein R₁, R₂, R₃, R₄ are the same as or different from one another and are independently selected from among H, C₁-C₅ alkyl, C₆-C₁₀ aryl, and C₃-C₆ cycloalkyl;

### (E) Amidine Ligands of the Formula

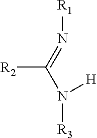

wherein R₁, R₂, R₃ are the same as or different from one another and are independently selected from among H, C₁-C₅ alkyl, C₆-C₁₀ aryl, and C₃-C₆ cycloalkyl;

### (F) Cyclopentadiene Ligands of the Formula

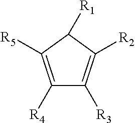

wherein R₁, R₂, R₃, R₄, R₅ are the same as or different from one another and are independently selected from among H, C₁-C₆ alkyl, C₆-C₁₀ aryl, C₁-C₈ alkoxy, C₁-C₈ alkylsilyl, and pendant ligands with additional functional group(s) that can provide further coordination to the metal center, e.g., —CH₂—CH₂—N(CH₃)₂;

### (G) Betadiketimine Ligands of the Formula

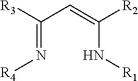

wherein R₁, R₂, R₃, R₄ are the same as or different from one another and are independently selected from among C₁-C₆ alkyl, C₆-C₁₀ aryl, silyl and C₁-C₈ alkylamine; and

### (H) Amine Ligands of the Formula

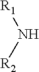

wherein R₁, R₂ are the same as or different from one another and are independently selected from among C₁-C₅ alkyl, C₆-C₁₀ aryl, and C₃-C₆ cycloalkyl.

Other aspects, features and advantages of the invention will be more fully apparent from the ensuing disclosure and appended claims.

### BRIEF DESCRIPTION OF THE DRAWINGS

FIG. 1 is a thermal ellipsoid plot of a strontium guanidinate of the invention.

### DETAILED DESCRIPTION OF THE INVENTION

Previous strontium and barium complexes used in ALD processes have required high (>300° C.) transport temperatures and have resulted in non-conformal surface coverage. The non-conformal coverage has been attributed to formation of oligomeric species during complex decomposition on the substrate surface during the ALD process.

The present inventors have discovered that utilizing sterically demanding ligands will allow for transport temperatures of less than or equal to 300° C. and that the sterically demanding nature of the ligand limits oligomerization behavior, promoting conformal film production in the ALD and RVD processes. Such ligands include aminotroponiminate, bis-oxazolinate and guanidinate ligands. While aminitroponiminate and bis-oxazolinate ligands have been discussed in the art, it has been with respect to Group III and lanthanide chemistry, not for CVD/ALD/RVD applications. (See Piers et al. Coord. Chem. Rev. vol. 233-4 p. 131-155 (2002)). A strontium guanidinate complex has also been reported in the art, but that compound has not been used for CVD/ALD/RVD applications. (See Feil, et al. Eur. J. Inorg. Chem. 2005 (21) p. 4438-4443 (2005)). The ligand precursors, metal source precursors and corresponding compositions of the invention are volatile and sufficiently stable precursors for CVD, ALD and RVD processes and are reactive at reasonable temperatures for those processes.

### Metal Aminotroponiminates

The present invention relates to metal aminotroponiminate ligand precursors, metal source precursors and compositions for use in CVD, ALD and RVD processes, and to methods of making the same.

In one aspect the invention relates to a ligand precursor of the formula:

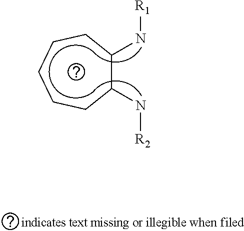

wherein R₁ and R₂ may be the same as or different from one another and each is independently selected from the group consisting of: H, C₁-C₅ alkyls, C₆-C₁₀ aryls and C₃-C₆ cycloalkyls. The term “C₁-C₅ alkyls” as used herein includes, but is not limited to, methyl, ethyl, propyl, isopropyl, butyl, s-butyl, t-butyl, pentyl and isopentyl and the like. The term “C₆-C₁₀ aryls” as used herein includes hydrocarbons derived from benzene or a benzene derivative that are unsaturated aromatic carbocyclic groups of from 6 to 10 carbon atoms. The aryls may have a single or multiple rings. The term “aryl” as used herein also includes substituted aryls. Examples include, but are not limited to phenyl, naphthyl, xylene, phenylethane, substituted phenyl, substituted naphthyl, substituted xylene, substituted phenylethane and the like. The term “C₃-C₆ cycloalkyls” as used herein includes, but is not limited to cyclopropyl, cyclobutyl, cyclopentyl, cyclohexyl and the like. In all chemical formulae herein, a range of carbon numbers will be regarded as specifying a sequence of consecutive alternative carbon-containing moieties, including all moieties containing numbers of carbon atoms intermediate the endpoint values of carbon number in the specific range as well as moieties containing numbers of carbon atoms equal to an endpoint value of the specific range, e.g., C₁-C₆, is inclusive of C₁, C₂, C₃, C₄, C₅ and C₆, and each of such broader ranges may be further limitingly specified with reference to carbon numbers within such ranges, as sub-ranges thereof. Thus, for example, the range C₁-C₆ would be inclusive of and can be further limited by specification of sub-ranges such as C₁-C₃, C₁-C₄, C₂-C₆, C₄-C₆, etc. within the scope of the broader range.

In another aspect the invention relates to a ligand precursor of the formula:

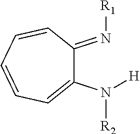

wherein R₁ and R₂ may be the same as or different from one another and each is independently selected from the group consisting of: H, C₁-C₅ alkyls, C₆-C₁₀ aryls and C₃-C₆ cycloalkyls.

In still another aspect, the invention relates to a metal source precursor of the formula:

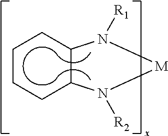

where R₁ and R₂ may be the same as or different from one another and each is independently selected from the group consisting of: H, C₁-C₅ alkyls, C₆-C₁₀ aryls and C₃-C₆ cycloalkyls. M is a metal selected from the group consisting of Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te and x is 1 to 8, dependent on the oxidation state of M. In one embodiment, M is barium or strontium. In another embodiment, the metal product may be bound by or coordinated to molecules of solvent. In still another embodiment, the metal product may be bound by or coordinated to molecules of additional ligands, such as, but not limited to, tetraglyme and pmdeta.

In another aspect, the invention provides a method of making a compound of the formula:

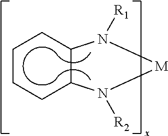

wherein R₁ and R₂ may be the same as or different from one another and each is independently selected from the group consisting of: H, C₁-C₅ alkyls, C₆-C₁₀ aryls and C₃-C₆ cycloalkyls. M is selected from the group consisting of Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te and x is 1 to 8, dependent on the oxidation state of M. In one embodiment, M is barium or strontium. In another embodiment, the metal product may be bound by or coordinated to molecules of solvent. In still another embodiment, the metal product may be bound by or coordinated to molecules of additional ligands, such as, but not limited to, tetraglyme and pmdeta.

In one aspect the method of making the compound where x=2 comprises the following reaction:

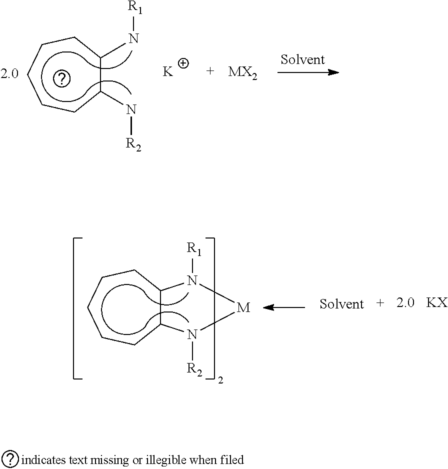

wherein X is selected from the group consisting of: chlorine, bromine and iodine. Where potassium is present in the reaction, one of skill could utilize other ions, as known in the art. Examples include, but are not limited to, alkali metals, such as sodium and lithium.

In another aspect the method of making the compound where x=2 comprises the following reaction:

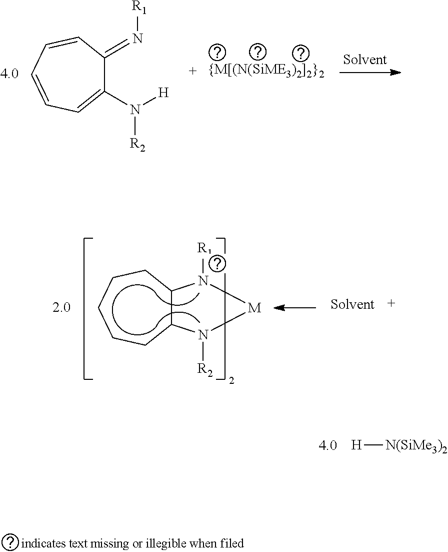

In various embodiments, the metal aminotroponiminate ligand precursors, metal source precursors and compositions thereof are utilized for CVD/ALD/RVD process applications.

In another aspect, the invention relates to a method of forming a metal-containing layer on a substrate. Such metals may include, but are not limited to, Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te. In a specific process embodiment, deposition of a metal layer on a substrate surface is carried out. In one embodiment, the metal is strontium or barium. The resulting layers can therefore include, without limitation, strontium titanate, barium titanate and strontium barium titanate.

### Metal Bis-Oxazolinates

The present invention relates in various embodiments to metal bis-oxazolinate ligand precursors, metal source precursors and compositions thereof for use in CVD, ALD and RVD processes, as well as methods of making the same.

In one aspect, the invention relates to a ligand precursor of the formula:

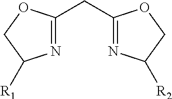

wherein R₁ and R₂ may be the same as or different from one another and each is independently selected from the group consisting of: H, C₁-C₅ alkyls, C₆-C₁₀ aryls and C₃-C₆ cycloalkyls.

In another aspect the invention provides a ligand precursor of the formula:

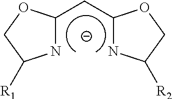

wherein R₁ and R₂ may be the same as or different from one another and each is independently selected from the group consisting of: H, C₁-C₅ alkyls, C₆-C₁₀ aryls and C₃-C₆ cycloalkyls.

In another aspect the invention relates to a metal source precursor of the formula:

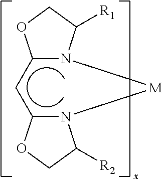

wherein each of the R₁ and R₂ substituents may be the same as or different from one another and each is independently selected from the group consisting of: H, C₁-C₅ alkyls, C₆-C₁₀ aryls and C₃-C₆ cycloalkyls. M is a metal selected from Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te and where x is 1 to 8, dependent on the oxidation state of M. In one embodiment, M is barium or strontium. In another embodiment, the metal product may be bound by or coordinated to molecules of solvent. In still another embodiment, the metal product may be bound by or coordinated to molecules of additional ligands, such as, but not limited to, tetraglyme and pmdeta.

In another aspect the invention provides a method of making a compound of the formula:

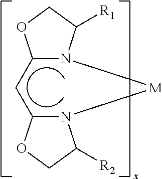

wherein each of the R₁ and R₂ substituents may be the same as or different from one another and each is independently selected from the group consisting of: H, C₁-C₅ alkyls, C₆-C₁₀ aryls and C₃-C₆ cycloalkyls. M is a metal selected from Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te and where x is 1 to 8, dependent on the oxidation state of M. In one embodiment, M is barium or strontium. In another embodiment, the metal product may be bound by or coordinated to molecules of solvent. In still another embodiment, the metal product may be bound by or coordinated to molecules of additional ligands, such as, but not limited to, tetraglyme and pmdeta.

In one aspect the method of making the compound where x=2 comprises the following reaction:

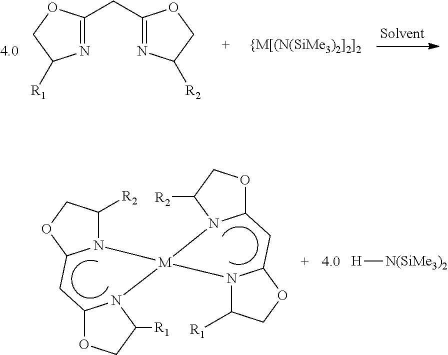

In another aspect the method of making the compound where x=2 comprises the following reaction:

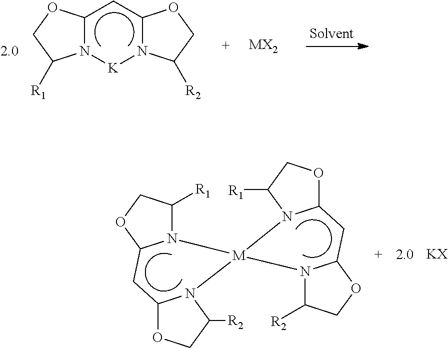

where X is selected from the group consisting of chlorine, bromine and iodine, and K is a potassium or sodium.

In one aspect, the metal bis-Oxazolinate ligand precursors, metal source precursors and compositions thereof are utilized for CVD/ALD/RVD processes. The invention in a specific aspect relates to a method of forming a metal containing layer on a substrate. Such metals include, without limitation, strontium and barium.

In a specific embodiment, the CVD/ALD/RVD process may include, but is not limited to, deposition of a metal layer on a substrate surface. Such metals may include, but are not limited to, Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te. In a specific process embodiment, deposition of a metal layer on a substrate surface is carried out. In one embodiment, the metal is strontium or barium. The resulting layers may include, but are not limited to strontium titanate, barium titanate and strontium barium titanate.

### Metal Guanidinates

The present inventors have also discovered that the use of sterically demanding guanidinate ligands generate homoleptic and monomeric strontium and barium complexes for use in CVD, ALD and RVD processes. These guanidinate ligands are utilized in homoleptic and monomeric precursors that are transportable (volatile) at temperatures specific to the ALD process. Additionally, the sterically demanding nature of the guanidinate ligands promotes conformal deposition of metals, such as barium or strontium, among others.

The present invention in a specific aspect relates to strontium and barium guanidinate ligand precursors, metal source precursors and compositions thereof for use in CVD, ALD and RVD processes and methods of making and using such precursors and compositions.

In one aspect the invention relates to a ligand precursor of the formula:

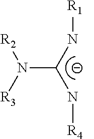

wherein R₁, R₂, R₃ and R₄ may be the same as or different from one another and each is independently selected from the group consisting of: H, C₁-C₅ alkyls, C₆-C₁₀ aryls and C₃-C₆ cycloalkyls.

In another aspect the invention relates to a ligand precursor of the formula:

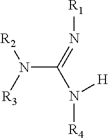

wherein R₁, R₂, R₃ and R₄ may be the same as or different from one another and each is independently selected from the group consisting of: H, C₁-C₅ alkyls, C₆-C₁₀ aryls and C₃-C₆ cycloalkyls.

In still another aspect the invention relates to a ligand precursor of the formula:

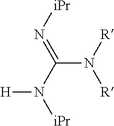

Wherein R′ may be the same or different from one another and may be methyl or iPr.

In yet another aspect the invention relates to a metal source precursor of the formula:

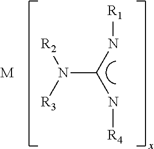

wherein R₁, R₂, R₃ and R₄ may be the same as or different from one another and each is independently selected from the group consisting of: H, C₁-C₅ alkyls, C₆-C₁₀ aryls and C₃-C₆ cycloalkyls. M is a metal selected from Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te and x is 1 to 8, dependent on the oxidation state of M. In one embodiment, M is barium or strontium. In another embodiment, the metal product can be bound by or coordinated to molecules of solvent. In still another embodiment, the metal product may be bound by or coordinated to molecules of additional ligands, such as, but not limited to, tetraglyme and pmdeta.

In still a further aspect, the invention relates to a method of making a compound of the formula:

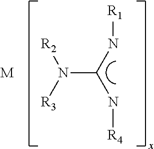

wherein R₁, R₂, R₃ and R₄ may be the same as or different from one another and each is independently selected from the group consisting of: H, C₁-C₅ alkyls, C₆-C₁₀ aryls and C₃-C₆ cycloalkyls. M is a metal selected from Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te and x is 1 to 8, dependent on the oxidation state of M. In one embodiment, M is barium or strontium. In another embodiment, the metal product may be bound by or coordinated to molecules of solvent. In still another embodiment, the metal product may be bound by or coordinated to molecules of additional ligands, such as, but not limited to, tetraglyme and pmdeta.

In one aspect the method of making the compound where x=2 comprises the following reaction:

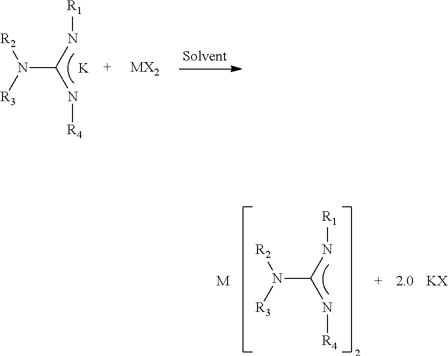

wherein X is selected from the group consisting of chlorine, bromine and iodine and K is selected from the group consisting of potassium and sodium.

In a further aspect the invention relates to a metal source precursor of the formula:

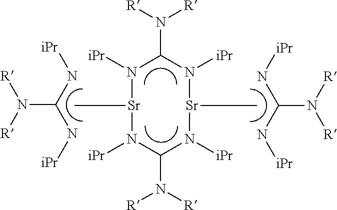

wherein R′ may be the same or different from one another and may be methyl or iPr.

In still a further aspect the invention relates to a method of making a compound of the formula:

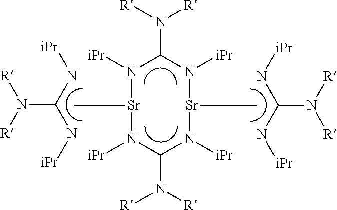

wherein R′ may be the same or different from one another and may be methyl or iPr.

In one aspect, the method of making the compound comprises the following reaction:

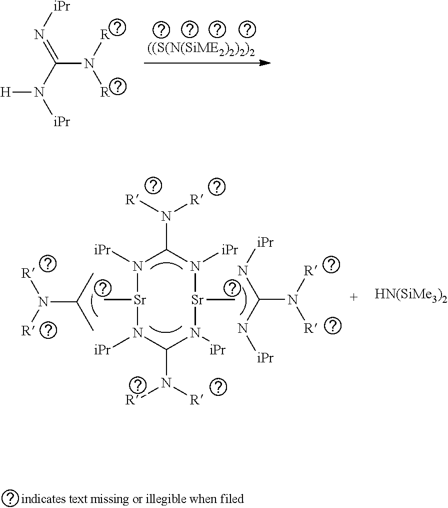

wherein R′ may be the same or different from one another and may be methyl or iPr.

In another aspect the invention relates to a metal source precursor of the formula:

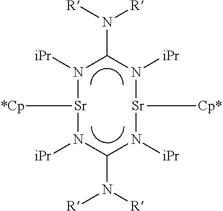

wherein R′ may be the same or different from one another and may be methyl or iPr and wherein Cp* is pentamethylcyclopentadienyl.

In still an additional aspect the invention relates to a method of making a compound of the formula:

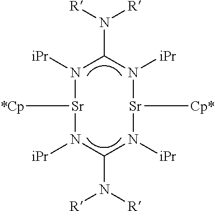

wherein R′ may be the same or different from one another and may be methyl or iPr and wherein Cp* is pentamethylcyclopentadienyl.

In one aspect, the method of making the compound comprises the following reaction:

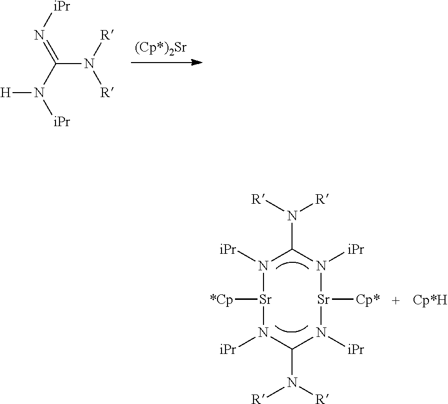

wherein R′ may be the same or different from one another and may be methyl or iPr and wherein Cp* is pentamethylcyclopentadienyl.

In another aspect the invention relates to a metal source precursor of the formula:

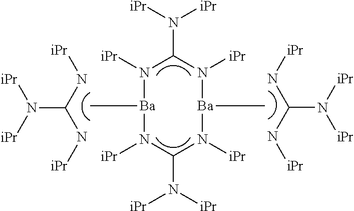

In an additional aspect the invention relates to a method of making a compound of the formula:

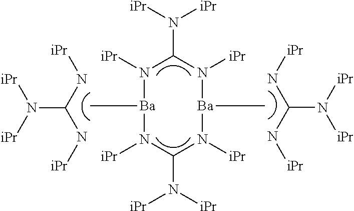

In still another aspect, the method of making the compound comprises the following reaction:

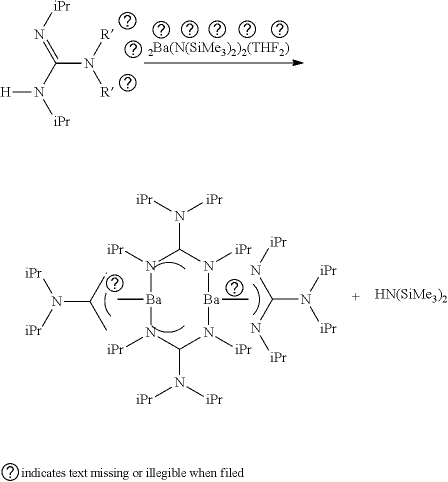

wherein R′ may be the same or different from one another and may be methyl

In a further aspect the invention relates to a metal source precursor of the formula:

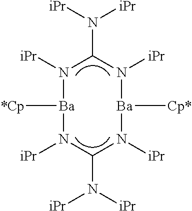

wherein Cp* is pentamethylcyclopentadienyl.

In still a further aspect the invention relates to a method of making a compound of the formula:

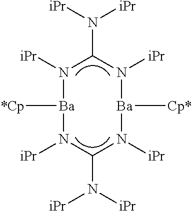

wherein Cp* is pentamethylcyclopentadienyl.

In another aspect, the method of making the compound comprises the following reaction:

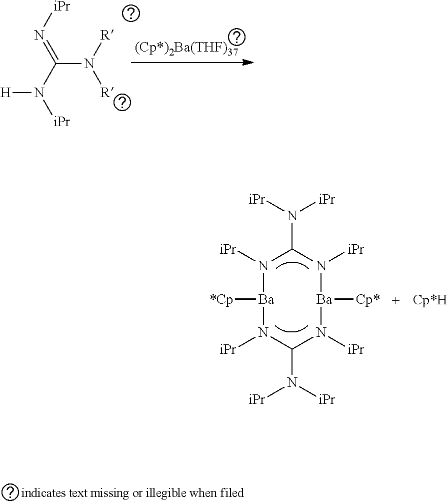

wherein R′ may be the same or different from one another and may be methyl or iPr and wherein Cp* is pentamethylcyclopentadienyl.

In one aspect, the metal guanidinate ligand precursors, metal source precursors and compositions thereof are utilized for CVD/ALD/RVD process applications. As such, another aspect of the invention relates to a method of forming a metal containing layer on a substrate. Such metals may include, but are not limited to, Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te. In a specific process embodiment, deposition of a metal layer on a substrate surface is carried out. In one embodiment, the metal is strontium or barium. The resulting layers can include, but are not limited to strontium titanate, barium titanate and strontium barium titanate.

### Guanidinate Ligands

The present invention in another aspect relates to guanidinate ligand precursors, metal source precursors and compositions thereof for use in CVD, ALD and RVD processes. The properties of complexes including guanidinate ligands are readily adjusted by varying the steric demands of the ligands.

In another aspect, the invention relates to a ligand precursor of the formula:

R₁N═C(NR₂R₃)NR₄M

wherein R₁, R₂, R₃ and R₄ may be the same as or different from one another and each is independently selected from the group consisting of: H, C₁-C₅ alkyls, C₆-C₁₀ aryls and C₃-C₆ cycloalkyls. M is selected from the group consisting of sodium and potassium.

In still another aspect the invention relates to a ligand precursor of the formula:

R₁N═C(NR₂R₃)NR₄H

wherein R₁, R₂, R₃ and R₄ may be the same as or different from one another and each is independently selected from the group consisting of: H, C₁-C₅ alkyls, C₆-C₁₀ aryls and C₃-C₆ cycloalkyls.

In still another aspect the invention provides a metal source precursor of the formula:

M[R₁N═C(NR₂R₃)NR₄]ₓ

wherein R₁, R₂, R₃ and R₄ may be the same as or different from one another and each is independently selected from the group consisting of: H, C₁-C₅ alkyls, C₆-C₁₀ aryls and C₃-C₆ cycloalkyls. M is selected from the group consisting of titanium, yttrium, zirconium, hafnium, praseodymium, erbium, ytterbium, lanthanum, niobium, tantalum, molybdenum, tungsten, ruthenium, osmium, calcium, strontium, barium, iridium, cobalt, nickel, palladium, platinum, copper, silver, gold, zinc, cadmium, gallium, aluminum, germanium, indium, tin, lead, antimony, bismuth, magnesium, europium, and tellurium. X is and x is 1 to 8, dependent on the oxidation state of M.

In various embodiments, the guanidinate ligand precursors, metal source precursors and compositions thereof are utilized in CVD/ALD/RVD processes. Such process may include, but is not limited to, deposition of a metal layer on a substrate surface. Another aspect of the invention is a method of forming a metal containing layer on a substrate. Such metals may include, but are not limited to titanium, yttrium, zirconium, hafnium, praseodymium, erbium, ytterbium, lanthanum, niobium, tantalum, molybdenum, tungsten, ruthenium, osmium, calcium, strontium, barium, iridium, cobalt, nickel, palladium, platinum, copper, silver, gold, zinc, cadmium, gallium, aluminum, germanium, indium, tin, lead, antimony, bismuth, magnesium, europium, and tellurium.

### DEFINITIONS AND ADDITIONAL DETAILS

The terms “complex” or “compound” as used herein is a substance made up of atoms of two or more elements. For example, an organometallic compound is a compound wherein a carbon is covalently bound to a metal. Other metal complexes and compounds are set forth herein. Complexes or compounds of the invention include ligand precursors and metal source precursors. The terms compound and complex are used interchangeably herein.

“Ligand” as used herein is a molecule or other chemical entity that binds to another molecule, e.g., molecule or ion that is covalently bound to a central metal atom to form an organometallic compound.

“Precursor” as used herein is a chemical entity that precedes and is the source of another chemical entity. A “ligand precursor” is a ligand starting material that is subsequently attached to a metal to form a metal source precursor for use in CVD, ALD and/or RVD applications. A “metal source precursor” is a compound that is usable for depositing metal on a substrate in a CVD, ALD or RVD process.

The novel ligand precursors, metal source precursors and compositions thereof, as described herein are usefully employed for forming thin films by CVD, ALD and/or RVD processes, utilizing process conditions, including appertaining temperatures, pressures, concentrations, flow rates and CVD, ALD and/or RVD techniques, as readily determinable within the skill of the art for a specific application, based on the disclosure herein.

In CVD, ALD and/or RVD usage, the metal source precursors of the invention are volatilized to form a precursor vapor that is then contacted with a microelectronic device substrate under elevated temperature vapor decomposition conditions to deposit a metal on the substrate.

CVD involves the contacting of a volatile metal-organic compound in the gas phase with areas of a substrate where growth of a metal film is required (e.g., for formation of an interconnect). A surface catalyzed chemical reaction, e.g., thermal decomposition, occurs and produces deposition of the desired metal. Since the metal film progressively grows on the desired surface, the resulting film is of a uniform thickness and highly conformal even to severe (e.g., high aspect) geometries. CVD is well suited to use in fabricating submicron high aspect ratio features.

ALD involves the deposition of successive monolayers over a substrate within a deposition chamber that is typically maintained at subatmospheric pressure. An exemplary method includes feeding a single source precursor into a deposition chamber to form a first monolayer on a substrate disposed therein. Thereafter, the flow of the first source precursor is terminated and an inert purge gas, e.g., nitrogen or argon, is flowed through the chamber to exhaust any unreacted first source precursor from the chamber. Subsequently, a second source precursor, which may be the same as or different from the first metal source precursor, is flowed into the chamber and reacts with the above-mentioned adsorbed mono-layer precursor materials on the substrate, forming a monolayer. The above process can be repeated until a layer of desired thickness and composition has been formed on the substrate.

RVD, like ALD, involves deposition of successive monolayers over a substrate. An exemplary method includes feeding a single source precursor into a deposition chamber to form a first substantially saturated monolayer on a substrate surface. Thereafter, the flow of the first deposition metal source precursor is terminated and an inert purge gas, e.g., nitrogen or argon, is flowed through the chamber to exhaust any unreacted first source precursor and/or any byproducts from the chamber. Subsequently, a second source precursor is flowed into the chamber to form a second monolayer on the first monolayer. The second monolayer in specific embodiments can react with the first monolayer, and in other embodiments the second monolayer is non-reactively deposited on the first monolayer. An additional source precursor can form a successive monolayer, or the above process can be repeated until a layer of desired thickness and composition has been formed on the substrate.

The metal source precursors of the invention are volatile and thermally stable, and are usefully employed as CVD, ALD and/or RVD precursors under reduced pressure deposition conditions in corresponding CVD, ALD or RVD reactors.

The compositions of the present invention can be delivered to the CVD, ALD or RVD reactors in a variety of ways. For example, a liquid delivery system may be utilized, with the solid precursor(s) being dissolved in organic solvents, and liquid delivery processes being used to meter the solution into a vaporizer for transport of the vapor to the reactor. Alternatively, a combined liquid delivery and flash vaporization process unit may be employed, to enable low volatility materials to be volumetrically delivered, so that reproducible transport and deposition are achieved without thermal decomposition of the precursor, in order to provide a commercially acceptable CVD, ALD or RVD process. In still another alternative, a liquid delivery system may be utilized wherein the precursor is stored in and delivered from an ionic liquid.

In liquid delivery formulations, metal source precursors that are liquids may be used in neat liquid form, or liquid or solid metal source precursors may be employed in solvent formulations containing same. Thus, metal source precursor formulations of the invention may include solvent component(s) of suitable character as may be desirable and advantageous in a given end use application to form metals on a substrate.

Suitable solvents may for example include alkane solvents (e.g., hexane, heptane, octane, and pentane), aryl solvents (e.g., benzene or toluene), amines (e.g., triethylamine, tert-butylamine), imines and carbodiimides (e.g., N,N′-diisopropylcarbodiimide) alcohols, ethers, ketones, aldehydes and the like. The utility of specific solvent compositions for particular metal source precursors may be readily empirically determined, to select an appropriate single component or multiple component solvent medium for the liquid delivery vaporization and transport of the specific metal source precursor that is employed.

In another aspect of the invention, a stabilizing ligand may be added to the CVD, ALD or RVD reactors before, concurrent with or after addition of the metal source precursors. Such ligands may include, but are not limited to tetraglyme and pmdeta.

In another aspect of the invention, a solid delivery system may be utilized, for example, using the ProE-Vap® solid delivery and vaporizer unit (commercially available from ATMI, Inc., Danbury, Conn., USA).

In another aspect of the invention, a liquid delivery system may be utilized, for example using the NOWTrak® system (commercially available from ATMI, Inc., Danbury, Conn., USA). In still another aspect of the invention, the packaging utilized in liquid delivery employing the NOWTrak® system includes a disposable liner adapted to hold the liquid precursor composition. Exemplary systems include, but are not limited to, those set forth in U.S. Pat. No. 6,879,876, filed Jun. 13, 2001 and issued Apr. 12, 2005 and titled “Liquid handling system with electronic information storage”; U.S. patent application Ser. No. 10/139,104, filed May 3, 2002 and titled “Liquid handling system with electronic information storage”; U.S. patent application Ser. No. 10/742,125, filed Dec. 19, 2003 and titled “Secure Reader System”; and U.S. Provisional Patent Application No. 60/819,681 filed Jul. 10, 2006 entitled “Fluid storage vessel management systems and methods employing electronic information storage,” all of which are hereby incorporated by reference in their entirety.

The metal source precursors of the invention may be packaged in a precursor storage and dispensing package of any suitable type. Depending on the form, e.g., solid or liquid form, of the precursor, preferred precursor storage and dispensing packages include those described in U.S. Provisional Patent Application No. 60/662,515 filed in the names of Paul J. Marganski, et al. for “SYSTEM FOR DELIVERY OF REAGENTS FROM SOLID SOURCES THEREOF” and the storage and dispensing apparatus variously described in U.S. Pat. No. 5,518,528; U.S. Pat. No. 5,704,965; U.S. Pat. No. 5,704,967; U.S. Pat. No. 5,707,424; U.S. Pat. No. 6,101,816; U.S. Pat. No. 6,089,027; U.S. Patent Application Publication 20040206241; U.S. Pat. No. 6,921,062; U.S. patent application Ser. No. 10/858,509; and U.S. patent application Ser. No. 10/022,298.

A wide variety of CVD, ALD or RVD process conditions may be employed in the use of the metal source precursors of the present invention. Generalized process conditions in specific embodiments include substrate temperatures in a range of 150-400° C., preferably 150-300 and more preferably less than or equal to 300° C.; pressure in a range of 0.05-5 Torr; carrier gas flows of helium, hydrogen, nitrogen, or argon in a range of 25-750 sccm; and vaporizer temperatures in a range of 50 to 180° C.

The invention in a further aspect relates to mixed ligand barium or strontium complexes suitable for use in CVD, ALD and RVD applications. Such mixed ligand barium or strontium complexes have the general formula:

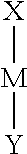

wherein M is barium or strontium, X and Y are each monoanionic and selected from the parent ligands (A)-(H) below, with the proviso that X and Y are different from one another:
(A) Triazacyclononane-Amide (tacn) Ligands of the Formula

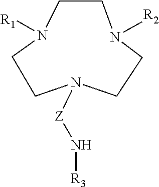

wherein: Z is (CH₂)₂ or SiMe₂; and R₁, R₂ and R₃ are the same as or different from one another, and each is independently selected from among C₁-C₅ alkyl, C₆-C₁₀ aryl, and C₃-C₆ cycloalkyl;

### (B) Aminotroponimine Ligands of the Formula

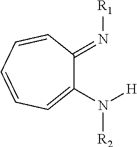

wherein R₁, R₂ are the same as or different from one another and each is independently selected from among H, C₁-C₅ alkyl, C₆-C₁₀ aryl, and C₃-C₆ cycloalkyl;

### (C) Bis(Oxazole) Ligands of the Formula

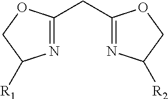

wherein R₁, R₂ are the same as or different from one another and each is independently selected from among H, C₁-C₅ alkyl, C₆-C₁₀ aryl, and C₃-C₆ cycloalkyl;

### (D) Guanidine Ligands of the Formula

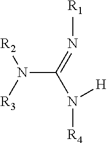

wherein R₁, R₂, R₃, R₄ are the same as or different from one another and are independently selected from among H, C₁-C₅ alkyl, C₆-C₁₀ aryl, and C₃-C₆ cycloalkyl;

### (E) Amidine Ligands of the Formula

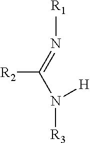

wherein R₁, R₂, R₃ are the same as or different from one another and are independently selected from among H, C₁-C₅ alkyl, C₆-C₁₀ aryl, and C₃-C₆ cycloalkyl;

### (F) Cyclopentadiene Ligands of the Formula

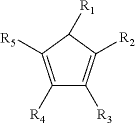

wherein R₁, R₂, R₃, R₄, R₅ are the same as or different from one another and are independently selected from among H, C₁-C₆ alkyl, C₆-C₁₀ aryl, C₁-C₈ alkoxy, C₁-C₈ alkylsilyl, or pendant ligands with additional functional group(s), which can provide further coordination to the metal center, e.g., —CH₂—CH₂—N(CH₃)₂;

### (G) Betadiketimine Ligands of the Formula

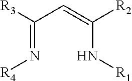

wherein R₁, R₂, R₃, R₄ are the same as or different from one another and are independently selected from among C₁-C₆ alkyl, C₆-C₁₀ aryl, silyl and C₁-C₈ alkylamine; and

### (H) Amine Ligands of the Formula

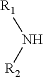

wherein R₁, R₂ are the same as or different from one another and are independently selected from among C₁-C₅ alkyl, C₆-C₁₀ aryl, and C₃-C₆ cycloalkyl.

The foregoing mixed ligand barium or strontium complexes are usefully employed for deposition of conformal barium- or strontium-containing films using CVD/ALD/RVD techniques, as monomeric barium or strontium precursors that are transportable (volatile) at temperatures specific to such processes. This aspect of the invention utilizes sterically demanding ligands to generate mixed-ligand, monomeric barium or strontium complexes suitable for CVD/ALD/RVD, in which the ligands are selected from tacn (A), aminotroponimines (B), bis-oxazolines (C), guanidines (D), amidines (E), cyclopentadienes (F), beta-diketimines (G), and amines (H). Such ligands will exist in their monoanionic form once associated with the metal. The sterically demanding ligands are selected to force monomeric structures enabling compound transportation at low temperatures.

The mixed ligand complexes of the invention can be readily synthesized from the parent ligands and the metal, wherein each of the two coordinated ligands is different from one another in the complex. Such mixed ligand complexes can be utilized as reagents for barium or strontium deposition in CVD, ALD or RVD processes conducted at relatively low temperatures.

The following examples are intended to illustrate, but not limit the invention.

### Example 1

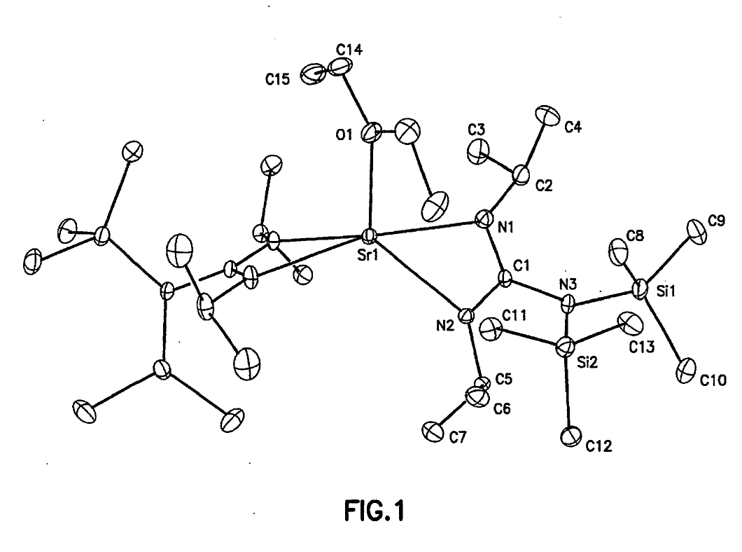

A non-limiting example for the synthesis of strontium guanidinate Sr{(i-pr)NC{N(SiMe₃)₂}N(i-pr)}₂.(Et₂O) is described below. To a stirring 20 ml ether suspension of SrI₂ (1.00 g, 2.93 mmol) was added Na{(i-pr)NC{N(SiMe₃)₂}N(i-pr)} (1.811 g, 5.85 mmol). The mixture was stirred for 8 days and filtered through a 0.2 micron filter. The resulting filtrate was concentrated under reduced pressure to afford 1.33 grams of Sr{(i-pr)NC{N(SiMe₃)₂}N(i-pr)}₂.(Et₂O). Single crystals of Sr{(i-pr)NC{N(SiMe₃)₂}N(i-pr)}₂.(Et₂O) were grown from a concentrated pentane solution at −30° C. and an X-ray crystallographic study was carried out on a single crystal. The thermal ellipsoid plot is shown in FIG. 1.

### Example 2

Another non-limiting example for the synthesis of strontium guanidinate [Sr(iPrNC(NMe₂)NiPr)₂]₂ is described here. To a toluene or benzene solution of {Sr[N(SiMe₃)₂]₂}₂ (0.31 g, 0.38 mmol, 10 ml solvent) was added 4.0 eq of iPrN(H)C(NMe₂)=NiPr (0.26 g, 1.50 mmol). On standing X-ray quality crystals of [Sr(iPrNC(NMe₂)NiPr)₂]₂ formed overnight and were isolated by filtration in 69% yield.

Although the invention has been described with reference to the above descriptions and examples, it will be understood that modifications and variations are encompassed within the spirit and scope of the invention. Accordingly, the invention is limited only by the following claims.

1-39. (canceled)

40. A composition comprising a compound or complex selected from the group consisting of: 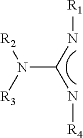
(A) compounds of the formula:
wherein R₁, R₂, R₃ and R₄ may be the same as or different from one another and each is independently selected from the group consisting of: H, C₁-C₅ alkyls, C₆-C₁₀ aryls and C₃-C₆ cycloalkyls;
(B) compounds of the formula: 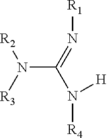
wherein R₁, R₂, R₃ and R₄ may be the same as or different from one another and each is independently selected from the group consisting of: H, C₁-C₅ alkyls, C₆-C₁₀ aryls and C₃-C₆ cycloalkyls; and
(C) compounds of the formula: 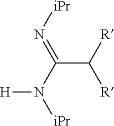
wherein R′ may be the same or different from one another and may be methyl or iPr;
(D) compounds of the formula: 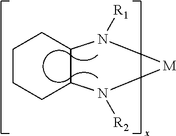
wherein:
R₁ and R₂ may be the same as or different from one another and each is independently selected from the group consisting of: H, C₁-C₅ alkyls, C₆-C₁₀ aryls and C₃-C₆ cycloalkyls;
M is selected from the group consisting of Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te; and x is 1 to 8, dependent on the oxidation state of M;
(E) compounds of the formula: 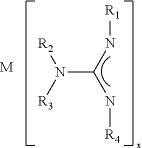
wherein:
R₁, R₂, R₃ and R₄ may be the same as or different from one another and each is independently selected from the group consisting of: H, C₁-C₅ alkyls, C₆-C₁₀ aryls and C₃-C₆ cycloalkyls; M is selected from the group consisting of Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te; and x is 1 to 8, dependent on the oxidation state of M;
(F) compounds of the formula: 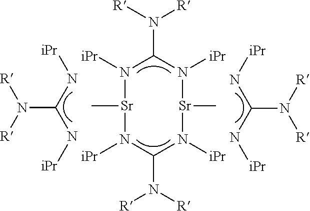
wherein R′ may be the same or different from one another and may be methyl or iPr;
(G) compounds of the formula: 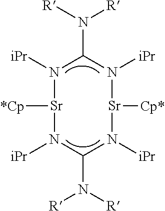
wherein R′ may be the same or different from one another and may be methyl or iPr and wherein Cp* is pentamethylcyclopentadienyl;
(H) compounds of the formula: 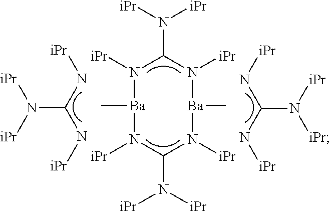
(I) compounds of the formula: 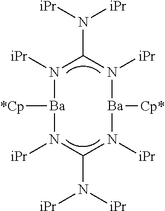
wherein Cp* is pentamethylcyclopentadienyl;
(J) compounds of the formula:
R₁N═C(NR₂R₃)NR₄M
wherein:
R₁, R₂, R₃ and R₄ may be the same as or different from one another and each is independently selected from the group consisting of: H, C₁-C₅ alkyls, C₆-C₁₀ aryls and C₃-C₆ cycloalkyls; and
M is selected from the group consisting of Na and K; and
(K) compounds of the formula:
R₁N═C(NR₂R₃)NR₄H
wherein:
R₁, R₂, R₃ and R₄ may be the same as or different from one another and each is independently selected from the group consisting of: H, C₁-C₅ alkyls, C₆-C₁₀ aryls and C₃-C₆ cycloalkyls.
(L) compounds of the formula:
M[R₁N═C(NR₂R₃)NR₄]ₓ
wherein:
R₁, R₂, R₃ and R₄ may be the same as or different from one another and each is independently selected from the group consisting of: H, C₁-C₅ alkyls, C₆-C₁₀ aryls and C₃-C₆ cycloalkyls;
M is selected from the group consisting of Ti, Y, Zr, Hf, Pr, Er, Yb, La, Nb, Ta, Mo, W, Ru, Os, Ca, Sr, Ba, Ir, Co, Ni, Pd, Pt, Cu, Ag, Au, Zn, Cd, Ga, Al, Ge, In, Sn, Pb, Sb, Bi, Mg, Eu, and Te; and
x is the oxidation state of M; and
(M) mixed ligand barium or strontium complexes having the general formula: 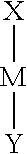
wherein M is barium or strontium, and X and Y are each monoanionic and selected from the parent ligands (i)-(vi) below, with the proviso that X and Y are different from one another:
(i) triazacyclononane-amide (tacn) ligands of the formula 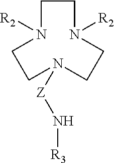
wherein: Z is (CH₂)₂ or SiMe₂; and R₁, R₂ and R₃ are the same as or different from one another, and each is independently selected from among C₁-C₅ alkyl, C₆-C₁₀ aryl, and C₃-C₆ cycloalkyl;
(ii) guanidine ligands of the formula 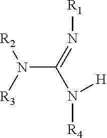
wherein R₁, R₂, R₃, R₄ are the same as or different from one another and are independently selected from among H, C₁-C₅ alkyl, C₆-C₁₀ aryl, and C₃-C₆ cycloalkyl;
(iii) amidine ligands of the formula 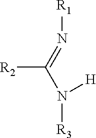
wherein R₁, R₂, R₃ are the same as or different from one another and are independently selected from among H, C₁-C₅ alkyl, C₆-C₁₀ aryl, and C₃-C₆ cycloalkyl;
(iv) cyclopentadiene ligands of the formula 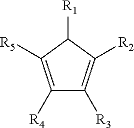
wherein R₁, R₂, R₃, R₄, R₅ are the same as or different from one another and are independently selected from among H, C₁-C₆ alkyl, C₆-C₁₀ aryl, C₁-C₈ alkoxy, C₁-C₈ alkylsilyl, and pendant ligands with additional functional group(s) that can provide further coordination to the metal center;
(v) betadiketimine ligands of the formula 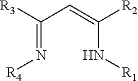
wherein R₁, R₂, R₃, R₄ are the same as or different from one another and are independently selected from among C₁-C₆ alkyl, C₆-C₁₀ aryl, silyl and C₁-C₈ alkylamine; and
(vi) amine ligands of the formula 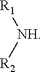
wherein R₁, R₂ are the same as or different from one another and are independently selected from among C₁-C₅ alkyl, C₆-C₁₀ aryl, and C₃-C₆ cycloalkyl.

41. The composition of claim 1, wherein the compound or complex is a compound of the formula (E) wherein M is barium.

42. The composition of claim 1, wherein the compound or complex is a compound of the formula (E) wherein M is strontium.

43. The composition of claim 1, wherein the compound or complex is a compound of the formula (J).

44. The composition of claim 1, wherein the compound or complex is a compound of the formula (K).

45. The composition of claim 1, wherein the compound or complex is a compound of the formula (L).

46. The composition of claim 1, wherein the compound or complex is a compound of the formula (M).

47. The composition of claim 1, wherein the compound or complex is a compound selected from the group consisting of the compounds of formula (J), (K) and (L).

48. The composition of claim 1, further comprising a solvent medium containing the compound or complex.

49. A method of depositing a metal layer on a substrate surface by chemical vapor deposition, atomic layer deposition or rapid layer deposition, comprising carrying out said deposition using a composition as claimed in claim 1.

50. The method of claim 49, comprising depositing the metal layer on a substrate surface by atomic layer deposition at temperature of less than or equal to 300 degrees Celsius.

51. The method of claim 49, comprising solution delivery or solid delivery of the composition.

52. The method of claim 49, wherein the metal layer is selected from the group comprising strontium titanate, barium titanate and strontium barium titanate.

53. The method of claim 49, wherein the compound or complex is a compound of the formula (E) wherein M is barium.

54. The method of claim 49, wherein the compound or complex is a compound of the formula (E) wherein M is strontium.

55. The method of claim 49, wherein the compound or complex is a compound of the formula (J).

56. The method of claim 49, wherein the compound or complex is a compound of the formula (K).

57. The method of claim 49, wherein the compound or complex is a compound of the formula (L).

58. The method of claim 49, wherein the compound or complex is a compound of the formula (M).

59. A substrate coated with one or more film monolayers of one or more metals, obtained by a method selected from chemical vapor deposition, atomic layer deposition and rapid vapor deposition, wherein at least one precursor includes a composition as claimed in claim 1.
